(19) United States
(12) Patent Application Publication
(10) Pub. No.: US 2019/0029936 A1
(43) Pub. Date: Jan. 31, 2019

(54) SULFATE-FREE, NATURALLY DERIVED, POWDER SHAMPOO

(71) Applicant: OWA Haircare, Inc., New York, NY (US)

(72) Inventor: Kailey R. BRADT, Big Flats, NY (US)

(73) Assignee: OWA Haircare, Inc., New York, NY (US)

(21) Appl. No.: 16/045,168

(22) Filed: Jul. 25, 2018

Related U.S. Application Data
(60) Provisional application No. 62/537,237, filed on Jul. 26, 2017.

(51) Int. Cl. A61K 8/41; A61K 8/46; A61K 8/92; A61K 8/73; A61K 8/365; A61K 8/44; A61K 8/19; A61K 8/02; A61Q 5/02; A61Q 5/12

(52) U.S. Cl. CPC A61K 8/416; A61K 8/463; A61K 8/922; A61K 8/731; A61K 8/732; A61K 8/365; A61K 2800/48; A61K 8/19; A61K 8/022; A61Q 5/02; A61Q 5/12; A61K 2800/30; A61K 8/44

(57) ABSTRACT

A sulfate-free, naturally derived, powder which includes a primary surfactant, a thickening agent, at least one of a conditioning agent, and wherein the powdered shampoo is free of sulfates and is naturally derived. A method of using the powder shampoo.

### CROSS-REFERENCE TO RELATED APPLICATIONS

This application claims the benefit of priority under 35 U.S.C. § 119(e) to U.S. Provisional Application No. 62/537,237, filed 26 Jul. 2017, and entitled SULFATE-FREE, NATURALLY DERIVED, POWDER SHAMPOO, which is incorporated herein by reference in its entirety.

### FIELD OF THE INVENTION

The present invention relates to compositions and methods of making a sulfate-free, naturally derived powder shampoo that hydrates upon contact with water. More particularly, the shampoo powder of the present invention, upon contact with water and shear stresses, forms a liquid that does not compromise lather or scent, creating a mild and conditioning shampoo.

### BACKGROUND

Shampoo is typically sold as a liquid, which is made up mostly of water, in some cases up to 80% water by weight. Liquid shampoos are inconvenient for travel, and fluids are often restricted on flights. Additionally, liquid shampoos are frequently manufactured by environmentally unsustainable methods and the distribution methods pose environmental risks. For instance, due to the use and waste of plastic from packaging, the use and transportation of fresh water from the manufacturing area, and excessive use of fuel and energy necessary for manufacturing and transportation, shampoo production tends to be environmentally unfriendly. Accordingly, a need exists to promote and maintain an environmentally friendly solution to this industry in which there is a need to reduce waste, increase efficiency, and promote a green supply chain.

### SUMMARY

This invention relates to a sulfate-free, naturally derived powdered shampoo, which overcomes the shortcomings of the prior art and provides additional advantages. This shampoo powder readily forms a liquid shampoo when combined with water and minimal shear stress, such as the shear stress of rubbing hands together. The resulting liquid shampoo is comparable to that of a traditional liquid shampoo in that it can contain all the features that can be found in a traditional liquid shampoo.

Disclosed is a powdered shampoo that, in one aspect, includes: a primary surfactant, a thickening agent, at least one conditioning agent, and wherein the powdered shampoo is free of sulfates and naturally derived.

Also disclosed is a method, the method including providing a powdered shampoo, applying an amount of powdered shampoo to a user's hands, rubbing the amount of powdered shampoo between the user's hands under running water to reach a desired lather, applying the wet powdered shampoo to the user's hair, massaging the wet powdered shampoo into the user's skin and hair, and rinsing the wet powdered shampoo out of the user's hair.

### DETAILED DESCRIPTION

Descriptions of well-known materials, fabrication tools, processing techniques, etc., are omitted so as to not unnecessarily obscure the invention in detail. It should be understood, however, that the detailed description and the specific examples, while indicating embodiments of the invention, are given by way of illustration only, and are not by way of limitation. Various substitutions, modifications, additions and/or arrangements within the spirit and/or scope of the underlying inventive concepts will be apparent to those skilled in the art from this disclosure.

Generally stated, disclosed herein is a sulfate-free, naturally derived powder shampoo that hydrates upon contact with water. The sulfate-free, naturally derived, shampoo powder of the present invention, upon contact with water and minimal shear stress, forms a liquid that does not compromise lather or scent as compared to traditional liquid shampoos, creating a mild and conditioning shampoo from a dry powder.

Currently, consumers have a large selection of liquid shampoos, however there is an absence of shampoo powder that can achieve the same features as that of a traditional liquid shampoo. While reetha powder or paste, made from soap not powder, exists, it does not have the same properties as modern shampoo. The present invention discloses a sulfate-free, naturally derived powder shampoo option, which, upon contact with water, hydrates to form a traditional liquid shampoo with cleansing properties, lather, and other features, from the hydration of the shampoo, as well as through the use of optional additives such as ingredients that moisturize the scalp to decrease dandruff and conditioning agents to reduce frizz, in some embodiments.

According to one or more embodiment, some or all of the ingredients used in the preparation of the shampoo powder of this disclosure should possess good water solubility, meaning that upon contact with water, some or all of the ingredients will be soluble. It has been found that the thickeners used in the preparation of the shampoo powder of this invention should thicken with minimal shear stresses, for example, by a rubbing motion of the hands together manually without external force.

Some definitions are provided to assist in the explanations below:

By “sulfate-free” it is meant that there are no sulfate containing surfactants and/or other ingredients, for example sodium lauryl sulfate, sodium laureth sulfate, ammonium lauryl sulfate, and the like salts or ethers of sulfuric acid that contain the anion SO₄²⁻or the —OSO₂O— group.

By “naturally derived” it is meant that ingredients are, as the term is considered by the FDA for food labeling, “natural” to mean “nothing artificial or synthetic.”

By “mild” it is meant the cleansing agents in the shampoo powder are not sulfate containing compounds as defined by “sulfate-free” above.

By “conditioning” it is meant that the hair has improved manageability, and provides a soft feel to the hair after washing.

The present invention is concerned with the discovery that combining together components which may include at least one primary surfactant, at least one thickening agent, at least one conditioning agent and/or at least one slip agent, optionally at least one secondary surfactant, and optional additives, containing no sulfate-containing surfactants, all of a natural derivation, can be formulated into a shampoo powder, which, upon contact with water and shear stress, forms a liquid that does not compromise lather or scent, creating a mild and conditioning shampoo. By mild, the shampoo may be gentle on hair, including but not limited to having a pH of approximately 4.0 to 6.5, and in some embodiments, between approximately 4.5 and 5.5. The conditioning qualities include leaving hair soft and moisturized.

In some embodiments, a primary surfactant is used in a powdered shampoo according to the instant application. Surfactants, which are useful in the practice of the present invention, can include all non-sulfate containing detergents that are naturally derived, including but not limited to sodium lauryl methyl isethionate, sodium cocoyl isethionate, sapindus trifoliatus fruit extract, cocamidopropyl betaine, cetyl bentaine and disodium cocoamphodiacetate. These surfactants not only provide cleansing properties to the powdered shampoo, but also contain good lathering properties, and can also be considered as foam boosters for the powdered shampoo. In one embodiment, the primary surfactant may include sodium cocoyl isethionate, which is commercially available as a free-flowing powder from many sources, one source includes Innospec® as Pureact I-78. The amount of primary surfactant, including sodium cocoyl isethionate in some embodiments, to obtain the desired effect can be from approximately 25% to approximately 75% by weight. In some embodiments, the amount of primary surfactant can include approximately 30%, approximately 35%, approximately 40%, approximately 45%, approximately 50%, approximately 55%, approximately 60%, approximately 65%, or approximately 70. Throughout the application, approximately should be understood to include plus or minus 5% of the given values.

In some embodiments, a secondary surfactant can be utilized in conjunction with the primary surfactant. The secondary surfactant may include, but is not limited to, any of the above surfactants. In some embodiments, a secondary surfactant is cetyl bentaine, which is also commercially available. The amount of secondary surfactant, such as cetyl bentaine, to obtain the desired effect may include from approximately 1% to approximately 25% by weight. In some embodiments, the amount of secondary surfactant can include approximately 5%, approximately 10%, approximately 15%, or approximately 20%. The secondary surfactant should be a different surfactant than the primary surfactant, and may be chosen for its properties, which may be desired in the powdered shampoo but absent from the primary surfactant. Any of the surfactants utilized may be in a dry form, and in some embodiments, include a free-flowing powder.

Thickening agents which are useful in the practice of the present invention include thickeners in which good water solubility is possessed and minimal shear stresses from rubbing hands can thicken the shampoo powder and water mixture to obtain the desirable viscosity of the liquid shampoo including, but not limited to, hydroxypropyl starch phosphate, cellulose gum, xanthan gum, tapioca starch, and guar gum. In some embodiments, the thickening agent is cellulose gum, which is commercially available as a free-flowing powder from many sources, one preferred source is supplied by CP Kelco® as Cekol 30000. The amount of thickening agent, such as cellulose gum, to obtain the desired effect may include from approximately 5% to approximately 30% by weight. In some embodiments, the amount of secondary surfactant can include approximately 10%, approximately 15%, approximately 20%, or approximately 25%. The water solubility of the thickeners, as well as the minimal shear stress require to activate thickeners, aids in the ability to form a good lather, equivalent to that of liquid shampoos. It is understood that one or a plurality of thickening agents may be utilized. The desired viscosity of the hydrated shampoo can be between approximately 1,000 cP and approximately 10,000 cP as measured at 10 rpm at room temperature. In some embodiments, the viscosity may be approximately 2,000 cP, approximately 3,000 cP approximately 4,000 cP, approximately 5,000 cP approximately 6,000 cP, approximately 7,000 cP approximately 8,000 cP, or approximately 9,000 cP.

Conditioning agents and/or slip agents may be useful in the practice of the present invention for increasing the wellness aspects of powdered shampoos according to some embodiments. For instance, conditioning agents and/or slipping agents may include conditioning agents in which good water solubility is possessed, including but not limited to plant derived proteins, plant extracts, and quaterniums. In some embodiments, the conditioning agent may include hydrolyzed rice protein, which is commercially available as a free-flowing powder from many sources, such as Seiwa Kasei® as Promois WR-SP. The amount of conditioning and/or slipping agent, in some embodiments rice protein, necessary to obtain the desired effect may include from about 0.1% to 10% by weight. In some embodiments, the amount of conditioning agent, which may include a slip agent, can include approximately 0.5%, approximately 1%, approximately 1.5%, approximately 2%, approximately 2.5%, approximately 3%, approximately 3.5%, approximately 4%, approximately 5%, approximately 5.5%, approximately 6%, approximately 6.5%, approximately 7%, approximately 7.5%, approximately 8%, approximately 8.5%, approximately 9%, or approximately 9%. Additionally, a plurality of conditioning agents may be used, with or without a separate slip agent.

The sulfate-free, naturally derived, powdered shampoo, according to some embodiments of the present invention, may further include one or more optional additives. Additives can include, for instance, chelating agents, pH adjustors, preservatives, fragrances, and colorants. When these optional additives are present in the sulfate-free, naturally derived, shampoo powder, they may range from 0.1% to 30% by weight. In some embodiments, the amount of total additives can include approximately 1%, approximately 5%, approximately 10%, approximately 15%, approximately 20%, or approximately 25%. In some embodiments, one or more chelating agents can be utilized, which can include, for example, trisodium ethylenediamine disuccinate, sodium citrate. In some embodiments, one or more pH adjusters can be utilized, which can include, for example, sodium bicarbonate, sodium hydroxide, and L-arginine. Additionally, fragrances may be utilized, which may include any naturally-derived fragrances, including but not limited to essential oils. In some embodiments, one or more colorants may be utilized, which can include rose hips, clays, and tea leaves, among other natural colorants.

In some embodiments, the optional additives may not be supplied in a dry form like the other ingredients, so long as the final shampoo powder is dry following the addition of the optional ingredients. The dry ingredients may be capable of absorbing any moisture and still resulting in a completely or substantially dry shampoo powder, or the solvent of the additive may be capable of evaporating during manufacturing and/or mixing of the powdered shampoo. Substantially dry can include less than approximately 5% moisture in some embodiments, and in further embodiments can include less than 4%, less than 3%, less than 2%, less than 1%, or completely dry in some embodiments.

The powdered shampoo of the invention can be prepared, generally, by combining the free flowing powders together in a suitable container to create a powder mixture of said ingredients. Generally, as used throughout, powder can refer to any bulk solid, fine or course, including but not limited to dust, granules, and granulations. The term free flowing refers to the flowability of the powder, specifically to its ability to flow in the processing steps required to mix the powdered shampoo.

Non-free flowing powders can be added to the mixture so long as the final product maintains the appearance and properties of the described present invention. Non-free flowing powders can include essential oils and solids ingredients in a solvent such as glycerin or an alcohol and other solids that can provide exfoliating properties and do not exhibit water solubility. The final mixture can be obtained by any appropriate methods. In some embodiments, a milling machine may be utilized for preparing powders, as well as for mixing the ingredients. A ribbon blender may be utilized for mixing the ingredients. The blended mixture may be passed though a sieve.

Once mixed, the powdered shampoo is completely or nearly completely dry. The powdered shampoo is capable of exhibiting lather and can function as a liquid shampoo when combined with water and shear stress. The water need not be any specific temperature to form the liquid shampoo in the hands, that is the powder will work with hot water, cold water, or any combination thereof. Water may be added to an amount of powdered shampoo in approximately 0.1 gram to approximately 1.0 gram of dry shampoo with any water desired. The water can include only what is present on a user's wet hands with no additional water, in some embodiments. It should be noted that the water can be added to the powdered shampoo directly in the palms of one's hands, in some embodiments, or by adding water to the shampoo in the hair, in other embodiments. The powdered shampoo lathers and is capable of lathering without agitation by shaking or being otherwise mixed together in a separate container, without centrifugation or other processes where external shear forces are needed to create the lathering or forming a liquid shampoo. That is, the shear stress, or shear force, provided by rubbing hands together, manually, is all that is required for creating a useable liquid shampoo, with no need to premix any further ingredients, nor requiring any extra containers for shaking or mixing ingredients or solvents. The shear stress provided by the rubbing motion of hands is sufficient to provide adequate lather, and mixing the ingredients together to provide the scent from any fragrance which may have been added, and mixing any colorants sufficiently.

In some embodiments, the powdered shampoo is free of certain ingredients which are considered less sustainable or which are harmful. For instance, the powdered shampoo can be free of sulfates, artificial colors, artificial fragrances, silicones, phthalates, animal-derived products, including any raw material of animal origin, parabens, and ingredients which have been tested on animals.

In some embodiments, the powdered shampoo can be analyzed for naturalness using an index combining the origin and denaturation of the raw materials. For instance, an origin index, similar to that developed by L'Oreal®, has a scale of 1 to 5. Any animal origin material receives a score of 1, fossil based material a score of 2, any mineral based material earns a score of 3, farmed plant material gets a score of 4, and organic or wild plant material gets a score of 5. Raw material of plant origin includes any material for which its percentage of carbon being plant origin exceeds 50% of total carbons. Such a rating takes into account renewable carbon and can be analyzed using ¹⁴C radiocarbon analysis. Thus, materials averaging over a threshold can be considered of desirable origin. In some embodiments, powdered shampoo according to the embodiments disclosed above, can include an origin index of 3.8 or higher, and in some embodiments, may include an origin index of 4.0, 4.2, 4.4, or 4.6.

Disclosed herein is also a method, the method including methods of using a powdered shampoo as disclosed above. The method, in some embodiments, includes providing a powdered shampoo according to any of the above embodiments, applying an amount of powdered shampoo to a user's hands. The amount of shampoo can include the specified weights disclosed above. The powdered shampoo is then rubbed between the user's hands, in some embodiments, just in wet hands. In some embodiments, the shampoo powder is rubbed between the hands under water, with an amount of water, or under running water, in order to reach a desired lather. This is largely a visual amount of lather and can vary by user, but generally includes a foam value of between approximately 50 mL and approximately 250 mL of foam following rubbing of the shampoo. In some embodiments, approximately 55 mL, approximately 60 mL, approximately 65, approximately 70, approximately 75 mL, approximately 80 mL, approximately 85 mL, approximately 90 mL, approximately 95 mL, approximately 100 mL, approximately 105 mL, approximately 110 mL, approximately 115 mL, approximately 120 mL, approximately 125 mL, approximately 130 mL, approximately 135 mL, approximately 140 mL, approximately 145 mL, approximately 150 mL, approximately 155 mL, approximately 160 mL, approximately 165 mL, approximately 170 mL, approximately 175 mL, approximately 180 mL, approximately 185 mL, approximately 190 mL, approximately 195 mL, approximately 200 mL, approximately 205 mL, approximately 210 mL, approximately 215 mL, approximately 220 mL, approximately 225 mL, approximately 230 mL, approximately 235 mL, approximately 240 mL, or approximately 245 mL of foam may be acquire from rubbing the powdered shampoo with water.

The powdered shampoo is then applied to the user's hair and massaged into the user's skin and hair for a period of time consisting of between approximately 20 seconds and approximately 10 minutes. The period of time can include, for instance, between approximately 30 seconds and 5 minutes, and approximately 1 minute and 2 minutes. In some embodiments, more water may be added to the shampoo by adding water to the hair with the shampoo already applied, allowing for control over the lather before and during use of the shampoo. The shampoo is then rinsed out of the user's hair.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs.

We claim:

1. A powdered shampoo comprising:
a primary surfactant;
a thickening agent;
at least one conditioning agent; and
wherein the powdered shampoo is free of sulfates and naturally derived.

2. The powdered shampoo of claim 1, wherein the powdered shampoo exhibits lather under the shear stress applied manually by hands.

3. The powdered shampoo of claim 1, wherein the primary surfactant comprises approximately 25% to approximately 75% by weight of the formulation.

4. The powdered shampoo of claim 3, wherein the primary surfactant comprises one of a group including: sodium lauryl methyl isethionate, sodium cocoyl isethionate, sapindus trifoliatus fruit extract, cocamidopropyl betaine, cetyl bentaine and disodium cocoamphodiacetate.

5. The powdered shampoo of claim 4, wherein the primary surfactant comprises sodium lauryl methyl isethionate.

6. The powdered shampoo of claim 1, wherein the thickening agent comprises approximately 5% to approximately 30% by weight of the formulation.

7. The powdered shampoo of claim 6, wherein the thickening agent comprises at least one of a group including: hydroxypropyl starch phosphate, cellulose gum, xanthan gum, and guar gum.

8. The powdered shampoo of claim 1, wherein the at least one conditioning agent comprises approximately 0.1% to approximately 10% by weight of the formulation.

9. The powdered shampoo of claim 8, wherein the at least one conditioning agent comprises at least one of a group including: plant derived proteins, plant extracts, and quaterniums.

10. The powdered shampoo of claim 9, wherein the at least one conditioning agent comprises hydrolyzed rice protein.

11. The powdered shampoo of claim 1, further comprising:
a secondary surfactant.

12. The powdered shampoo of claim 11, wherein the secondary surfactant comprises approximately 1% to approximately 25% by weight of the formulation.

13. The powdered shampoo of claim 12, wherein the secondary surfactant comprises one of a group including: sodium lauryl methyl isethionate, sodium cocoyl isethionate, sapindus trifoliatus fruit extract, cocamidopropyl betaine, cetyl bentaine and disodium cocoamphodiacetate, and wherein the secondary surfactant is different than the primary surfactant.

14. The powdered shampoo of claim 1, further comprising:
at least one additive.

15. The powdered shampoo of claim 1, wherein the at least one additive in total comprises approximately 0.1% to approximately 30% by weight of the formulation.

16. The powdered shampoo of claim 15, wherein the at least one additive includes a chelating agent, wherein the chelating agent includes at least one of a group including:
trisodium ethylenediamine disuccinate, and sodium citrate.

17. The powdered shampoo of claim 15, wherein the at least one additive includes a pH adjuster, wherein the pH adjustor includes at least one of a group including: sodium bicarbonate, sodium hydroxide, and L-Arginine.

18. The powdered shampoo of claim 15, wherein the at least one additive includes a fragrance, wherein the fragrance includes essential oils.

19. The powdered shampoo of claim 15, wherein the at least one additive includes a colorant, wherein the colorant includes at least one of a group including: sodium bicarbonate, sodium hydroxide, and L-Arginine.

20. A method comprising:
providing a powdered shampoo according to claim 1;
applying an amount of powdered shampoo to a user's hands;
rubbing the amount of powdered shampoo between the user's hands;
applying the wet powdered shampoo to the user's hair;
massaging the wet powdered shampoo into the user's skin and hair; and
rinsing the wet powdered shampoo out of the user's hair.
